(19) United States
(12) Patent Application Publication
(10) Pub. No.: US 2025/0360059 A1
(43) Pub. Date: Nov. 27, 2025

(54) SOLID COSMETIC HAIR CARE COMPOSITION HAVING AT LEAST ONE CAVITY

(71) Applicant: CAPSUM, MARSEILLE (FR)

(72) Inventors: Sébastien BARDON, VEYRIER DU LAC (FR); Mathieu GOUTAYER, SAINT MALO (FR); Jean-François GONIDEC, AIX-EN-PROVENCE (FR)

(21) Appl. No.: 18/874,998

(22) PCT Filed: Jun. 28, 2023
(86) PCT No.: PCT/EP2023/067605

§ 371 Date: Dec. 13, 2024

(30) Foreign Application Priority Data
Jun. 28, 2022 (FR) FR2206462

(51) Int. Cl. A61K 8/02; A61Q 5/00

(52) U.S. Cl. CPC A61K 8/0216; A61Q 5/00

(57) ABSTRACT

A solid cosmetic hair and/or scalp care composition includes at least one hair care agent. The composition includes at least one cavity such that the composition has a porosity of at least 20% by volume relative to the total volume of the solid composition. The disclosure also relates to associated manufacturing processes.

The invention relates to a solid cosmetic composition for hair and/or scalp care.

The field of solid cosmetics generally concerns cleansing products for the skin or hair, in particular soaps or shampoos, or makeup products. In particular, solid cosmetics scarcely focus on the sector of hair and/or scalp care.

The solid products currently marketed are essentially in the form of sticks, powders to be dispersed or pods (or sheets) to be dispersed, generally based on water-soluble polyvinyl alcohol (PVA or PVOH).

However, such compositions are generally anhydrous and do not give full satisfaction.

Sticks generally display insufficient performance in term of homogeneity of deposit, comfort, sensory feeling, and/or hydration. These deposits are generally greasy and/or tacky. Also, use thereof implies repeated contact between the solid composition and the area to be treated which, for obvious reasons, not very hygienic. Additionally, degradation of sticks and/or breakage and/or disintegration thereof before they have been entirely used generates spoilage and is therefore wasteful.

With regard to powders to be dispersed, these are generally water-soluble or water-dispersible powders to which it is often difficult to add the proper amount of water to arrive at the final desired liquid composition. Also, the dispersion thereof in an aqueous medium is often perceived by users as being a disappointing experience since poor in quality. Finally, with this galenic form it is difficult to accommodate a fatty (or oily phase) and hence the presence of lipophilic ingredients. This at least partly explains why this type of composition is generally unsatisfactory from a sensory and efficacy viewpoint.

As for pods, this is an improper ecological solution since once dissolved in water, the polyvinyl alcohol pollutes groundwater. In addition, it is known that polyvinyl alcohol generally imparts a tacky, unpleasant texture.

In addition, solid cosmetic care compositions in single-dose form generally comprise much packaging, at least partly disposable and/or having properties that are not fully satisfactory in terms of prehension, ease of application, sensory feeling, hydration, and/or comfort on application.

The present invention sets out to overcome the aforementioned disadvantages by proposing solid cosmetic hair care compositions in particular in single-dose form, guaranteeing good prehension by the user, direct and comfortable application, requiring little packaging and allowing the presence of a fatty (or oily phase) and hence the presence of lipophilic ingredients.

### Composition

The invention therefore concerns a solid cosmetic hair and/or scalp care composition comprising at least one hair care agent, characterized in that said composition comprises at least one cavity such that said composition has porosity higher than or equal to 20%, preferably higher than or equal to 30%, better still higher than or equal to 40%, in particular higher than or equal to 50%, even higher than or equal to 60%, in particular higher than or equal to 70%, and most particularly higher than or equal to 80% by volume, relative to the total volume of said solid composition.

Therefore, a composition of the invention can still have a size and/or volume similar to those of current solid compositions, but differing through the presence of at least one cavity such that the porosity of said composition is higher than or equal to 20% by volume relative to the total volume of said composition.

By «solid» in the meaning of the invention, it is meant to designate a composition which, at ambient temperature and atmospheric pressure, will not collapse under its own weight. Advantageously, it is meant to designate any composition having a compression strength greater than or equal to 20 g, preferably greater than or equal to 50 g, and better still greater than or equal to 100 g, at ambient temperature (20-25° C.), after penetration by a cylinder probe of revolution having a diameter of 0.8 cm into the matrix of the composition to a depth of 1 mm at a rate of 0.5 mm/s and removal of said probe from the matrix of the composition at a rate of 0.5 mm/s, the compression strength being measured with an analyzer of “LFRA Texture Analyzer” type marketed by STEVENS/MECHTRIC.

A composition of the invention is not in the form of a paste. A composition of the invention is not a powdered composition and therefore not in powder form. Finally, a composition of the invention is not a porous solid either derived from agglomeration of powders. In a first embodiment, a composition of the invention can be an expanded composition and can therefore be in the form of a foam. In a second embodiment, a composition of the invention is not an expanded composition and is therefore not in the form of a foam.

A composition of the invention is not in the form of a mask.

Preferably, a solid composition of the invention is not coated with a water-soluble coating.

By «porosity» in the meaning of the present invention, it is meant to designate the assembly of voids in a solid material, these voids able to filled with fluids, water in particular, when the solid composition is placed in the presence of an aqueous solution e.g. water such as tap water.

By (cavity in the meaning of the present invention, it is meant to designate an empty space inside a solid body and which communicates directly with the outer medium similar to the cavities for example illustrated in FIGS. 1 to 3 and 6. A cavity can indifferently be termed by the words «orifice», «pore» or «hole». Advantageously, said cavity/cavities are through cavities i.e. the cavity designates an empty space inside a solid body and communicates directly with the outer medium either side of the solid body.

Therefore, the volume of said cavity/cavities in a solid composition of the invention represents at least 20%, in particular at least 30%, preferably at least 40%, in particular at least 50%, even at least 60%, in particular at least 70%, and most particularly at least 80% of the volume of said solid composition.

Advantageously, a solid composition of the invention has a density of between 0.2 and 0.8, preferably between 0.3 and 0.7, in particular between 0.4 and 0.6.

A solid composition of the invention can have a total volume of between 0.125 cm³ and 100 cm³, preferably between 0.250 cm³ and 75 cm³, in particular between 0.5 cm³ and 50 cm³, better still between 1 cm³ and 25 cm³, and more particularly between 2.5 cm³ and 15 cm³.

A solid composition of the invention can weigh between 0.025 g and 200 g, preferably between 0.25 g and 150 g, in particular between 0.5 g and 100 g, better still between 1 g and 75 g, more particularly between 2.5 g and 50 g, even between 5 g and 25 g («g» corresponding to the unit «gram»).

In a first preferred embodiment, a solid composition of the invention has a ratio between the volume of the cavities and the total volume of the composition of between 0.05 and 0.9, preferably between 0.1 and 0.8, in particular between 0.2 and 0.9, preferably between 0.2 and 0.8, better still between 0.2 and 0.7, even between 0.2 and 0.6.

By («total volume», it is meant to designate the volume resulting from the sum of the volume of the solid and the volume of the cavities.

In the present invention, the cavities can be said to be macroporous i.e. having pores of minimum width, even diameter, greater than or equal to 0.5 mm, preferably greater than or equal to 0.75 mm, and in particular greater than or equal to 1 mm, even between 0.5 mm and 2 cm, more particularly between 1 mm and 2 cm, preferably between 2 mm and 1 cm, in particular between 3 mm and 75 mm, better still between 4 mm and 50 mm and most particularly between 5 mm and 25 mm. In particular, the pores have a minimum width, even diameter of between 0.5 mm and 5 mm, preferably between 1 mm and 3 mm.

In this respect, the Applicant indicates that, at equivalent porosity, the fabrication of a stable solid composition is:
easy in the presence of cavities of small size, for example those contained in solid substrates in which the porosity results from inserting a gas during the production process, similar to the description given in U.S. Pat. No. 8,349,341, but
much more difficult in the presence of macroporosities such as those under consideration in the present invention.

The presence of cavities having the content and/or volume and/or size considered above, gives access to solid compositions that are less raw material-intensive whilst still maintaining satisfactory handling properties. A solid composition of the invention is therefore able to generate less loss. This weight gain has the further advantage of allowing less costly transport that is also less polluting. A solid composition of the invention is therefore advantageous both from an ecological and from an economic viewpoint.

Additionally, the inventors have observed an unexpected effect when a solid composition of the invention is placed in the presence of an aqueous phase. The inventors observed that a solid composition of the invention, when in contact with an aqueous phase, produces a greater quantity of foam and more rapidly than a solid composition of same size and volume but devoid of cavities, and even displays faster transformation kinetics between the original solid state into a liquid state e.g. of gel or cream type. Without wishing to be bound by any theory, it is the opinion of the inventors that the presence of cavities in the solid composition allows facilitated transition towards a viscous liquid composition, for example when the solid composition is mixed by hand, or allows an increased contact surface with the aqueous phase, in particular water, and hence better hydration of said solid composition.

Therefore, the invention even allows adjustment of the rate at which the solid composition will foam, swell and/or melt, by adjusting the developed surface area at constant volume, by adjusting percent porosity, and more particularly by acting on the shape and/or number and/or size of the cavities, even on the number of walls and/or thickness of the walls of the cavities.

Advantageously, the size and/or volume of the cavities can be adjusted to store and/or react the necessary amount of water to arrive at a final liquid composition having satisfactory/expected properties, even that are optimized and/or homogeneous (i.e. between different single doses) for example in terms of viscosity, hydration, comfort on application, and/or transformative effect.

With the composition of the invention, it is therefore possible to have access to evanescent compositions, namely able to change into liquid compositions in particular in the form of gels or creams, when in contact with an adequate amount of water.

Finally, the inventors have observed that the porous structure of a solid composition of the invention allows deposition of the composition on the hair and/or scalp that is more homogeneous, even without having recourse to means of application.

Preferably, a composition of the invention is a single-dose composition. This embodiment is particularly advantageous from a hygienic viewpoint in that it avoids repeated contacts between the solid composition and the hair and/or scalp. This embodiment, also allows easy adapting by the user to a desired cosmetic effect according to frame of mind (mood), style of dress, and/or time of day.

The invention therefore also concerns a packaging comprising several solid compositions of the invention in the form of single-doses of different care agents/cosmetic effect. The invention therefore concerns packaging comprising several solid compositions of the invention, preferably in single-dose form, said compositions comprising hair care agents and/or having contents of different hair care agent(s). This packaging therefore has the advantage of ensuring easy personalization of this care for the user according to frame of mind (mood), dress style, and/or time of day.

Preferably, a composition of the invention is a topical composition. A composition of the invention is not an oral composition.

A solid composition of the invention can be of any shape, for example spherical, ellipsoid, tetrahedral or polygonal, in particular in the form of a parallelogram, preferably a rectangular parallelogram.

Preferably, in particular to ensure good prehension, a solid composition of the invention has:
a width, or diameter, of between 1 cm and 10 cm, preferably between 2 cm and 8 cm, in particular between 3 cm and 6 cm, and better still between 4 cm and 5 cm; and/or
a height (or thickness) of between 0.5 cm and 5 cm, preferably between 1 cm and 4 cm, and in particular between 2 cm and 3 cm.

The cavity/cavities of a solid composition of the invention can be of any shape e.g. round, oblong, ovoid, triangular, polygonal, in particular hexagonal, honeycomb or tessellated (or partitioned) for example of Voroni diagram type, preferably of honeycomb shape such as illustrated in FIGS. 3 and 6.

The cavity/cavities of a solid composition of the invention can also represent letters or the logo of a trademark or company as illustrated in FIG. 2.

A solid composition of the invention comprises one or more cavities, preferably several cavities, the same or different.

In a first variant, a composition of the invention comprises at least one cavity, in particular a through cavity.

The cavities are through cavities, advantageously along an axis x-x′.

In a second preferred variant, a composition of the invention comprises several cavities, through cavities in particular, such as illustrated in FIGS. 3 and 6.

In one particular embodiment, a composition of the invention may comprise at least two cavities that are connected together. Said embodiment is advantageous since it can promote phenomena of capillarity at the time of contact with water, allowing an improvement in the aforementioned technical effects.

Advantageously, a solid composition of the invention comprises several cavities having a structure of honeycomb shape as illustrated in FIG. 3. This honeycomb shape is particularly advantageous in that it permits the manufacture of a solid composition having a ratio of «volume of cavity(cavities)/total volume of the composition» that is most favourable, in particular higher than or equal to 0.5, even higher than or equal to 0.75, without negatively affecting the prehension and robustness of said composition under mechanical stresses likely to be applied thereto and/or during storage. In other words, this honeycomb structure affords an excellent trade-off between cavity volume and mechanical strength.

Advantageously, the cavity/cavities of a solid composition of the invention comprise walls having a thickness of between 10 μm and 1500 μm, preferably between 20 μm and 1000 μm, in particular between 30 μm and 750 μm, more particularly between 50 μm and 500 μm, even between 100 μm and 400 μm. In particular, the cavity/cavities of a solid composition of the invention have walls of thickness between 250 μm and 500 μm.

Advantageously, a solid composition of the invention comprises several cavities provided with a structure of honeycomb shape having walls with a thickness of between 10 μm and 600 μm, preferably between 20 μm and 500 μm, in particular between 30 μm and 400 μm, more particularly between 50 μm and 300 μm, even between 100 μm and 200 μm.

In one particular embodiment, a composition of the invention may additionally comprise additional patterns separate from the cavities, said patterns possibly being obtained by embossing.

In a first variant, a composition of the invention is anhydrous. By «anhydrous» in the meaning of the present invention, it is meant to designate a composition which, before any contacting with water, has a water content of less than 5 weight %, preferably less than 1 weight % and in particular less than 0.5 weight %, relative to the total weight of said composition.

In a second variant, a composition of the invention comprises a continuous fatty phase and a dispersed aqueous phase. The composition of the invention is therefore in the form of an oily (or fatty) phase additionally comprising a least one dispersed aqueous phase, for example in the form of matrix spheres (or beads) or of core/shell type (or capsules), for example in the form of capsules as described in WO2010063937.

In one embodiment, the weight percentage of water in the dispersed aqueous phase can be between 0.5 and 50%, preferably between 1 and 40%, in particular between 2.5 and 30%, and better still between 5 and 20% by weight relative to the total weight of the composition.

The solid nature of a composition of the invention can be based on a phase mostly, even entirely, composed of a fatty phase comprising at least one lipophilic gelling agent (also called hereafter «solid fatty phase»).

Preferably, the solid nature of a composition of the invention is based on the presence of a fatty phase comprising at least one lipophilic gelling agent.

### Fatty Phase

The dispersed fatty phase of the composition of the invention has a melting point of between 50° C. and 100° C., preferably between 60° C. and 90° C.

The melting point of a fatty phase can be measured by differential scanning calorimetry (DSC), for example with the calorimeter sold under the trade name “DSC Q2000” by TA Instruments. The protocols for preparing samples and measurement are the following: a 5 mg sample to be tested, previously heated to 80° C. and sampled under magnetic stirring using a spatula that is also heated, is placed in a hermetically sealed aluminium capsule or sample pan. Two tests are conducted to ensure reproducibility of results. Measurements are taken on the calorimeter mentioned above. The oven is flushed with nitrogen. Cooling is ensured with an RCS 90 heat exchanger. The sample is brought to 20° C. and subjected to the following protocol: a first temperature rise from 20° C. to 130° C. at a heating rate of 5° C./minute, cooling from 130° C. to −80° C. at a cooling rate of 5° C./minute, and finally a second temperature rise from −80° C. to 130° C. at a heating rate of 5° C./minute. During the second temperature rise, the variation is measured in the difference between the energy absorbed by the empty sample pan and by the sample pan containing the sample, as a function of temperature. The melting point of the compound is the value of the temperature corresponding to the peak of the curve representing the variation in difference of absorbed energy as a function of temperature. The end melt temperature corresponds to the temperature at which 95% of the sample has melted.

### Lipophilic Gelling Agent

A composition of the invention comprises at least one lipophilic gelling agent.

The lipophilic gelling agent can be chosen from among organic or mineral, polymeric or molecular lipophilic gelling agents; fats solid at ambient temperature and pressure; and mixtures thereof.

Fats solid at ambient temperature and pressure are preferably chosen from among at least one wax, at least one pasty fat, at least one butter, and mixtures thereof.

Advantageously, a lipophilic gelling agent is a heat-sensitive gelling agent, namely one which reacts to heat, and in particular a gelling agent solid at ambient temperature and liquid at a temperature higher than 50° C., preferably higher than 60° C., better still higher than 70° C. Preferably, a heat-sensitive lipophilic gelling agent of the invention has a melting point of between 50° C. and 130° C., more preferably between 60° C. and 120° C.

As lipophilic gelling agent(s), mention can be made for example of those described in WO2021234135.

In one particularly preferred embodiment, the lipophilic gelling agent is chosen from among Castor Oil/IPDI Copolymer (and) Caprylic/Capric Triglyceride marketed in particular under the tradename Estogel M by PolymerExpert, Hydrogenated Castor Oil/Sebacic Acid Copolymer and the derivatives thereof, particularly those marketed under the tradenames Estogel Green (or Estogel G) and Estogel Green 40 by PolymerExpert, Caprylic/Capric Triglyceride (and) Polyurethane-79 in particular the one marketed under the tradename OILKEMIA™ 5S polymer by Lubrizol, Trihydroxystearin in particular as marketed under the tradename THIXCIN® R by Elementis Specialties, and mixtures thereof, and more preferably it is Castor Oil/IPDI Copolymer (and) Caprylic/Capric Triglyceride.

In particular, a composition of the invention may comprise from 10% to 90%, preferably from 20% to 80%, in particular from 30% to 70%, and better still from 40% to 60% by weight of lipophilic gelling agent(s) relative to the total weight of the composition.

### Oils

A composition of the invention may comprise at least one oil. By «oil» it is meant a fat that is liquid at ambient temperature and atmospheric pressure.

As oils of the invention, mention can be made for example of:
hydrocarbon oils of vegetable origin, such as described below;
hydrocarbon oils of animal origin such as perhydrosqualene and squalane;
synthetic esters and ethers, in particular of fatty acids, such as oils of formulas R¹COOR² and R¹OR² where R¹ represents the remainder of a C₈ to C₂₉ fatty acid, and R² represents a C₃ to C₃₀ branched or non-branched hydrocarbon chain such as Purcellin oil, isononyl isononanoate, isodecyl neopentanoate, isostearyl neopentanoate, isopropyl myristate, octyldodecyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl-malate, triisocetyl citrate, heptanoates, octanoates, fatty alcohol decanoates; polyol esters such as propylene glycol dioctanoate, neopentylglycol diheptanoate and diethyleneglycol diisononanoate; and the esters of pentaerythritol such as pentaerythrityl tetrabehenate (DUB PTB) or pentaerythrityl tetraisostearate (Prisorine 3631);
linear or branched hydrocarbons or mineral or synthetic origin, such as paraffin oils whether or not volatile and derivatives thereof, Vaseline, polydecenes, hydrogenated polyisobutene such as Parleam oil;
silicone oils, such as polydimethylsiloxanes (PDMS) for example whether or not volatile, having a linear or cyclic silicone chain, liquid or pasty at ambient temperature, in particular cyclopolydimethylsiloxanes (cyclomethicones) such as cyclohexasiloxane and cyclopentasiloxane; polydimethylsiloxanes (or dimethicones) comprising alkyl, alkoxy or phenyl groups either pendant or at the end of the silicone chain, groups having 2 to 24 carbon atoms; phenyl silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes;
fatty alcohols having 8 to 26 carbon atoms such as cetyl alcohol, stearyl alcohol and mixture thereof (cetylstearyl alcohol), or octyldodecanol;
partially hydrocarbon-based and/or silicone-based fluorinated oils such as those described in document JP-A-2-295912;
and mixtures thereof.

As oils, mention can be made for example of those described in WO2021234135.

A composition of the invention preferably comprises a least one vegetable oil e.g. copra oil, coconut oil, olive oil, sweet almond oil, castor oil, Babassu oil, avocado oil, and mixtures thereof.

In particular, a composition of the invention may comprise from 10% to 90%, preferably 20% to 80%, in particular 30% to 70%, and better still from 40% to 60% by weight of oil(s) relative to the total weight of the composition.

A composition of the invention may comprise at least one lipophilic gelling agent, preferably heat-sensitive, at least one oil, and mixtures thereof.

### Hair Care Agent

A solid composition of the invention, in particular the fatty phase and/or dispersed aqueous phase (if any) comprises at least one hair care agent.

As hair care agents of the invention, mention can be made of those usually used in care compositions for the hair and/or scalp.

In particular, as hair care agent of the invention, mention can be made of surfactants; fixative polymers such as those listed in applications FR2964565 or US2013289080; conditioning agents chosen in particular from among cationic conditioning agents; fats differing from the above-mentioned oils and lipophilic gelling agents; polysaccharides in particular chitosans and derivatives thereof; anti-UV agents (or UV filters) in particular those described in FR3041251; fillers such as nacreous filers, titanium dioxide, resins or clays, and mixtures thereof; fragrances (or scents); peptizing agents; acidic agents; alkaline agents, reducing agents, oxidizing agents; direct dyes or oxidative dyes; and mixtures thereof.

In particular, a solid composition of the invention comprises between 0.0001% and 60%, preferably between 0.001% and 50%, in particular between 0.01% and 40%, more particularly between 0.1% and 35%, and preferably between 1% and 25% by weight of hair care agents relative to the total weight of said composition.

In one embodiment, the solid hair care composition of the invention may comprise at least one surfactant, in particular a cationic, non-ionic, anionic, amphoteric surfactant and mixtures thereof. Said surfactants are chosen from among those usually used for hair care compositions.

As conditioning agents, mention can be made of cationic polymers, lipophilic in particular, preferably amodimethicone or an ingredient such as described in the patent application filed under no. FR2205170.

As conditioning agent, mention can also be made of amidoamine compounds. Examples of amidoamines useful in the compositions of the invention comprise the following but are not limited thereto: oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamidopropyl dimethylamine, cocamidopropyl dimethylamine, wheat germamidopropyl dimethylamine, sunflowerseedamidopropyl dimethylamine, almondamidopropyl dimethylamine, avocadamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, palmitamidopropyl dimethylamine, stearamidopropyl dimethylamine, linoleamidopropyl dimethylamine dimer dilinoleate, and mixtures thereof.

As Linoleamidopropyl Dimethylamine Dimer Dilinoleate, mention can be made for example of the raw material marketed by ALZO International Inc. under the tradename Necon LO-80.

In some other embodiments, the cationic conditioning agents can be chosen from among monoalkyl quaternary amines, dialkyl quaternary amines or polyquaternium compounds, or salts thereof.

For example, the cationic conditioning agents can be chosen from among Polyquaterium-10 (also called quaternized polyhydroxyethylcellulose), cetrimonium chloride, behentrimonium chloride, behentrimonium methosulfate, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, Quaternium-91, Polyquaternium-37 (e.g. under the tradename SALCARE), Quaternium-22, Quaternium-87, Polyquaternium-4, Polyquaternium-6, Polyquaternium-11, Polyquaternium-44, Polyquaternium-67, amodimethicone, lauryl betaine, Polyacrylate-1 Crosspolymer, steardimonium hydroxypropyl hydrolyzed wheat protein, behenamidopropyl PG-dimonium chloride, laurdimonium hydroxypropyl hydrolyzed soy protein, aminopropyl dimethicone, Quaternium-8, and dilinoleamidopropyl dimethylamine PEG-7 phosphate, and mixtures thereof.

In some embodiments, the cationic polymers can be chosen for example from the following: polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, or guar hydroxypropyltrimonium chloride, and mixtures thereof.

In one embodiment, the cationic conditioning agent is chosen from among Polyquaternium-67, Polyquaternium-10, Polyquaternium-37, or mixtures thereof. For example, Polyquaternium-37 is commercially available from BASF under the tradename SALCARE SC 96 (comprising Polyquaternium-37 (and) Propylene Glycol Dicaprylate/Dicaprate (and) PPG-1 Trideceth-6), and mixtures thereof

As fats, mention can be made for example of fatty alcohols, fatty esters, fatty acids, the derivatives thereof, or mixture thereof. More particularly the fat is:
a fatty ester chosen from among cetyl ester, Purcellin oil (cetearyl octanoate), isopropyl myristate, isopropyl palmitate, C12-C15, alkyl benzoate, 2-ethylphenyl benzoate, isopropyl lanolate, hexyl laurate, diisopropyl adipate, isononyl isononanoate, oleyl erucate, 2-ethylhexyl palmitate, isostearyl isostearate, diisopropyl sebacate, alcohol or polyalcohol octanoates, decanoates or ricinoleates, hydroxylated esters and pentaerythritol esters, and mixtures thereof;
a fatty alcohol chosen from among decyl alcohol, undecyl alcohol, dodecyl alcohol, myristyl alcohol, lauric alcohol, cetyl alcohol, stearyl alcohol, cetearyl alcohol, (cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol and mixtures thereof;
a polyol chosen from among ethylene glycol, propylene glycol, butylene glycol, hexylene glycol, pentylene glycol, diethylene glycol, dipropylene glycol, caprylyl glycol, 1,3-propanediol, glycerol, diglycerol, polyethylene glycols, and mixtures thereof; and
mixtures thereof.

As peptizing agents, acidic agents, alkaline agents, reducing agents or oxidizing agents, mention can be made of those usually used for hair and/or scalp care compositions.

As acidic agents, mention can be made for example of lactic acid and/or citric acid. As alkaline agents, sodium hydroxide can be cited for example or ammonia and/or arginine. As reducing agents, mention can be made for example of thioglycolic acid, thiolactic acid and/or thioglyoxilic acid. As oxidizing agents, mention can be made for example of hydrogen peroxide and/or urea peroxide.

### Additional Compounds

A composition of the invention, in particular the fatty phase, even the dispersed aqueous phase if any, may additionally comprise at least one additional compound differing from the above-mentioned lipophilic gelling agents, oils and hair care agents.

A composition of the invention may additionally comprise powders; glitter; colouring agents in particular whether or not water-soluble, whether or not liposoluble, organic or inorganic; materials with optical effect, liquid crystals, and mixtures thereof; particulate agents insoluble in the fatty phase; preserving agents; humectants; fillers with blur effect; effervescent agents; disintegrating agents (e.g. croscarmellose, carboxymethylcellulose, . . . ); diluents; disintegration promoters; stabilizers; chelating agents; emollients; modifying agents chosen from among texturizing agents (or gelling agents) and in particular hydrophilic gelling agents; modifiers of pH, of osmotic force, and/or refractive index modifiers etc., or any usual cosmetic additive; and mixtures thereof.

By «particulate agents insoluble in the fatty phase» in the meaning of the invention it is meant the group formed by pigments, ceramics, polymers in particular acrylic polymers, and mixtures thereof.

As hydrophilic gelling agents, mention can be made for example of those described in FR3041251.

A composition of the invention may additionally comprise at least one biological/cosmetic agent differing from the hair care agent. As biological/cosmetic agent, mention can be made of hydrating agents, vitamins, healing agents, antiwrinkle or anti-age agents, depigmenting agents, peeling agents, antioxidants, agents stimulating synthesis of dermal or epidermal macromolecules, skin relaxants, anti-perspirants, soothing agents and/or anti-age agents, and mixtures thereof.

Said biological/cosmetic agents are particularly described in more detail in FR3041251.

A composition of the invention may additionally comprise at least one solvent able to evaporate fully or partially during the production process as described below. As example of solvent, an apolar or scarcely polar organic solvent can be cited e.g. cyclohexane, THF (tetrahydrofuran), and mixtures thereof.

Advantageously, a solid composition of the invention is not composed of a network of fibres.

Evidently, those skilled in the art will take care to choose the additional raw materials, ingredient(s), agent(s) and/or compound(s) described above and/or the quantity thereof so that the advantageous properties of the product of the invention are not or are not substantially impaired by the envisaged addition. Also, skilled persons will ensure that the type and/or quantity of additional raw materials, ingredient(s), agent(s) and compound(s) described above are chosen as a function of the aqueous or fatty nature of the phase under consideration and/or having regard to the manufacturing process thereof. These adjustments lie within the general knowledge of persons skilled in the art.

The solid nature of a composition of the invention advantageously obviates the need for conventional packaging. The packaging for a solid composition of the invention can be restricted for example to a simple paper wrapping.

Advantageously, a solid composition of the invention is not composed of a network of fibres.

Preferably, a composition of the invention does not comprise an alcohol.

Preferably, a solid composition of the invention does not comprise polyvinyl alcohol (PVA or PVOH), a polyacrylate, a copolymer of acrylic acid and methacrylic acid, polyvinylidene fluoride (PVDF), polymethyl methacrylate (PMMA), polyvinylpyrrolidone, polyalkylene oxide, polyacrylonitrile (PES or PAN), polymethacrylate, polyethylene terephthalate, polyethylene, polypropylene, nylon, hyaluronic acid, starch and derivatives thereof, pullulan, gelatin, cellulose (e.g. rayon), hydroxypropylmethylcellulose, methylcellulose, carboxymethylcellulose, cotton, and one of the mixtures thereof.

A composition of the invention does not comprise (or is not based on) a water-soluble solid substrate e.g. of hydrogel type, in particular starch-based and optionally unstructured, and/or poloxamer 407.

### Manufacturing Process

A composition of the invention is obtained with any process known to skilled persons and adapted for the manufacture of a solid composition, subject to necessary adaptations to ensure the forming of cavities and hence of meeting the criterion of required porosity.

Preferably the process for manufacturing a solid composition of the invention does not comprise an aeration/injection step of a gas e.g. air, optionally followed by a forming step and drying.

### Process No. 1

In a first variant, a process for manufacturing a composition of the invention comprises at least the steps of:
(i) providing a composition in liquid or semi-liquid form;
(ii) pouring the composition into a mould and drying until a solid composition is obtained,
(iii) cutting the solid composition to the desired shape; and
(iv) forming cavities to obtain a solid composition of the invention.

Step (i) is advantageously performed under heat.

Advantageously, steps (iii) and (iv) are simultaneous.

Advantageously, steps (ii), (iii) and (iv) are simultaneous, in which case the shape of the mould is adapted to ensure forming of the cavity/cavities.

Step (iv) is advantageously conducted by means of a punch.

Process no. 1 is advantageous in that the offcuts obtained after step (iv) can be reused for further processing. These offcuts can be reheated and fed back into a subsequent no. 1 process at step (i) and/or (ii).

### Process No. 2

In a second variant, a process for the manufacture of a composition of the invention is an extrusion process.

Said manufacturing process of the invention comprises at least the steps of:
(a) providing a composition in liquid or semi-liquid form;
(b) passing the composition of step (a) through an extruding device equipped with a die (or die plate) of adapted shape to form a solid bar; and
(c) cutting the bar obtained at step (b) to obtain a solid composition of the invention.

For obvious reasons, step (b) is performed at a temperature adapted so that the composition is of sufficient viscosity and able to maintain the shape imparted by the die.

For obvious reasons, the shape of the die of the extruding device is also adapted to ensure formation of the cavity/cavities.

Advantageously, step (b) is conducted at a temperature lower than ambient temperature, for example at a temperature lower than or equal to 15° C., even lower than or equal to 10° C.

At step (b), the extruding device comprises a die having a shape adapted to form the desired cavity/cavities and desired porosity in the solid bar.

As an illustration, an extrusion process of the invention comprises a die such as the one illustrated in FIG. 4.

Step (c) can be performed using a knife or cutting wire, for example a butter cutting wire.

Preferably, steps (b) and (c) are simultaneous.

Preferably, the process for manufacturing a composition of the invention is the extrusion process no. 2 described above.

In one particular embodiment, applicable to processes no. 1 and no. 2 described previously, step (i)/(a) entails providing a fluid composition which may comprise at least one solvent able to evaporate fully or partially at step (ii)/(b), even also at step (iii) and/or (iv)/(c).

As solvent, mention can be made for example of an apolar or scarcely polar organic solvent e.g. cyclohexane, THF (tetrahydrofuran), and mixture thereof.

In processes no. 1 and no. 2 above, when the composition comprises at least one fatty phase comprising a least one lipophilic gelling agent, step (i)/(a) may necessitate heating said composition to a temperature higher than the highest melting point of the lipophilic gelling agents contained in said composition.

In one particular embodiment, the extrusion process no. 2 can be performed with a 3D printing technique.

### Use

A composition of the invention is dedicated to hair and/or scalp care.

In particular, a solid composition of the invention is not intended for washing hair and/or the scalp and is not therefore a soap of shampoo.

In particular, depending on whether the solid composition of the invention is intended for direct application to hair and/or the scalp, or necessitates prior mixing with an aqueous medium, a composition of the invention can for example be a cream, lotion, serum, hair colouring product (hair dye or bleach), a hair care product (lotions, creams, oils), a styling product (lotions, lacquers, brilliantines).

In a first embodiment, in particular when a composition of the invention is of single-dose type, a solid composition of the invention is intended to be placed in direct contact with the hair and/or scalp. Optionally, before application to hair and/or the scalp, the composition is mixed in the hands to ensure transition towards a viscous liquid composition.

In a second embodiment, a solid composition of the invention is intended to be placed in contact with an aqueous phase, water in particular, simultaneously with and/or after application to the area to be treated. When a composition of the invention is not of single-dose type, the contacting with water is performed on first use only, and supposes packaging of the «solid composition/water» mixture in a suitable container. For obvious reasons, this necessitates a solid composition having an adapted formula and a sufficient amount of water to ensure transition from a solid composition to a liquid composition, for example in gel form. This also necessitates the presence in the solid composition of at least one preserving agent to guarantee good storage over time of the liquid composition thus formed.

In one particular embodiment, a solid composition of the invention in particular when having a configuration of honeycomb type as previously described, may comprise a plurality of sections, polygonal sections in particular, each of the sections defining a fractionable perimeter to allow the separating of at least one section from the remainder of the solid composition. Therefore, in this embodiment, each section of the solid composition corresponds to a single dose.

Finally, the present invention also concerns a non-therapeutic cosmetic treatment method for hair and/or scalp care, comprising at least one step to apply at least one composition such as defined above to the hair and/or scalp.

In one particular embodiment, the present invention concerns a product comprising, or derived from the contacting between, at least one first solid composition as described above and at least one second liquid composition.

### First Solid Composition

In the meaning of the present invention, the first composition can indifferently be designated «first solid composition» or «solid composition», and corresponds to a solid composition as described above.

### Second Liquid Composition

In the meaning of the present invention, the second composition can indifferently be designated «second liquid composition» or «liquid composition».

In a first variant, the second composition is aqueous.

In a second variant, the second composition is anhydrous.

In a third variant, the second composition is an emulsion. An emulsion can be a direct (i.e. oil-in-water), invert (i.e. water-in-oil) or multiple emulsion, in particular a double emulsion (i.e. water-in-oil-in-water, oil-in-water-in-oil, or oil-in-oil-in-water).

In one particular embodiment, the second composition is an emulsion, preferably a stable, direct emulsion with high concentration of dispersed phase, and in particular it is an emulsion of HIPE type (i.e. «High Internal Phase Emulsion») or Apollonian emulsion.

A second composition of the invention is not in solid form at ambient temperature and ambient pressure i.e. it is able to flow under its own weight.

A second composition of the invention is in liquid form, preferably in gel form.

The viscosity of a second composition or at least the continuous phase thereof when in emulsion form, may vary extensively which allows varied textures to be obtained. Viscosity is measured at ambient temperature and ambient pressure following the method described in WO2017046305.

For obvious reasons, the viscosity of the second composition is adjusted to allow easy dispensing/application of the product of the invention.

In one embodiment, the viscosity of the second composition is advantageously from 5 000 mPa·s to 500 000 mPa·s, preferably from 10 000 mPa·s to 300 000 mPa·s, better still from 25 000 mPa·s to 100 000 mPa·s, and more particularly from 50 000 mPa·s to 75 000 mPa·s, as measured at 25° C. following the above-described method.

Preferably, the second composition can reduce the solidity of the first composition. In particular, the second composition can advantageously comprise at least one raw material able to reduce the solidity of the first composition. This embodiment is advantageous in that it allows the solid texture of the first composition to change into a softer texture. The term «transformative effect» can be used.

A product of the invention can advantageously have a weight ratio of «first composition(s)/second composition(s)» of between 0.01 and 10, preferably between 0.1 and 5, better still between 0.5 and 1.

In one particular embodiment, the first composition may have high contents of hair care agents such as described below. The first composition can therefore be termed a «booster».

In the light of the foregoing, a product of the invention is an extemporaneous product i.e. it is the user who, on first use of the product, will place the first composition in contact with the second composition.

The invention therefore also concerns a kit comprising:
at least one first solid composition as previously described;
at least one second liquid composition as previously described; and
optionally, at least one container.

A container can indifferently be designated a «container or» packaging or «primary packaging».

The invention further concerns use of the kit described above, to obtain a product of the invention.

Preferably, a kit of the invention may additionally comprise at least one application/dispensing means.

If the product is intended for cosmetic use, a kit of the invention may additionally comprise at least one means for application to keratin material, chosen for example from the group formed by spatulas, different brushes, puffs, or sponges.

In one particular variant, the kit may comprise several first compositions, the same or different, and/or several second compositions, the same or different, and/or at least one application means.

The invention therefore also concerns a kit comprising several first compositions in the form of single doses comprising hair care agents and/or having contents of different hair care agent(s).

Therefore, said kit of the invention advantageously allows easy adapting to the effect sought by users.

With regard to a cosmetic product, this embodiment enables a user to decide on the sought after cosmetic effect, in particular according to frame of mind (mood), dress style and/or time of day.

### DESCRIPTION OF THE FIGURES

FIG. 1 illustrates a composition of the invention of parallelepiped shape having a cavity of cylindrical shape. A corresponds to the side view, and B corresponds to the overhead view.

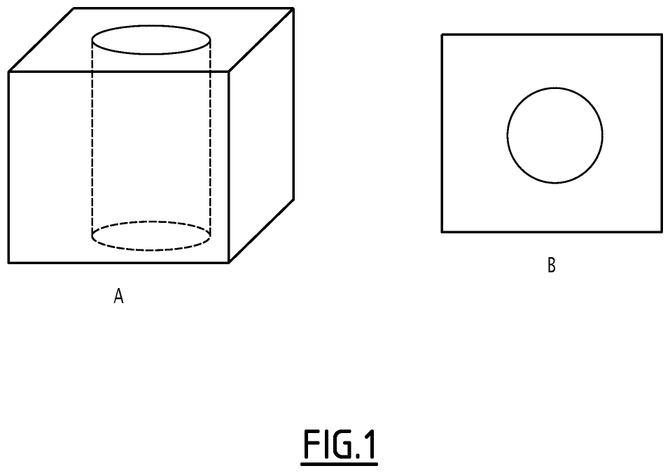

FIG. 2 illustrates a composition of the invention of ovoid shape having cavities (overhead view) representing the word «SOAP».

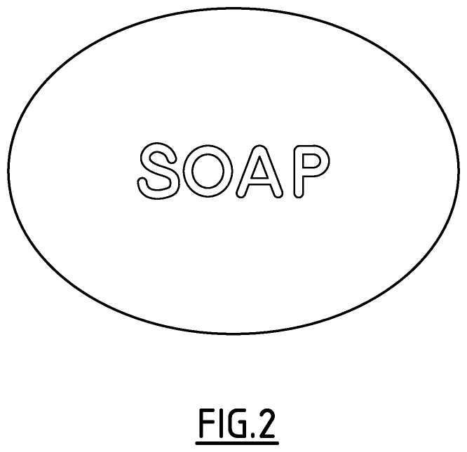

FIG. 3 illustrates a composition of the invention having honeycomb-shaped cavities (overhead view).

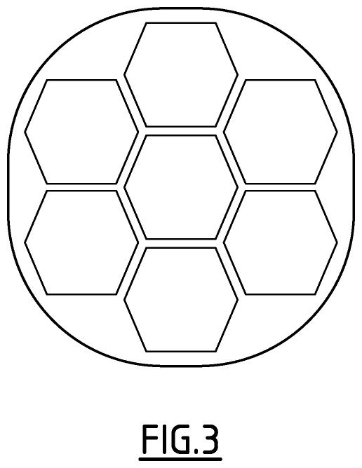

FIG. 4 illustrates a die used for the process of the invention.

FIG. 5 illustrates a comparative die.

FIG. 6 illustrates other examples of solid compositions of the invention.

### EXAMPLES

### Step 1—Manufacture:

TABLE 1

| NAME | INCI | % | Phase |
|---|---|---|---|
| LABRAFAC CC | Caprylic/CapricTriglyceride | q.s.* | A |
| CETIOL OE | Dicaprylyl Ether | 20.00 | A |
| JOJOBA OIL | Simmondsia Chinensis (Jojoba) | 8.00 | A |
| | Seed Oil | | |
| Avocado Oil | Persea Gratissima (Avocado) Oil | 4.00 | A |
| ESTOGEL M | Castor Oil/IPDI Copolymer (and) | 30.00 | A |
| | Caprylic/CapricTriglyceride | | |
| ESTOGEL | HydrogenatedCastor Oil/SebacicAcid | 8.00 | A |
| GREEN | Copolymer | | |
| REP'HAIR | Behenyl/Stearyl aminopropanediol | 0.20 | A |
| | esters | | |
| Coenzyme Q10 | Ubiquinone | 0.2 | B |
| COPHEROL | Tocopheryl Acetate | 0.50 | B |
| 1250 C | | | |
| FRAGRANCE | Fragrance | 0.50 | B |

| | |
|---|---|
| Total | 100.00 |

*in sufficient amount

The ingredients of phase A are placed in a container and the mixture is heated under agitation to 90° C. until homogenization.
The mixture is cooled to 70° C. and the ingredients of phase B are added under agitation until homogenization.

### Step 2—Extrusion:

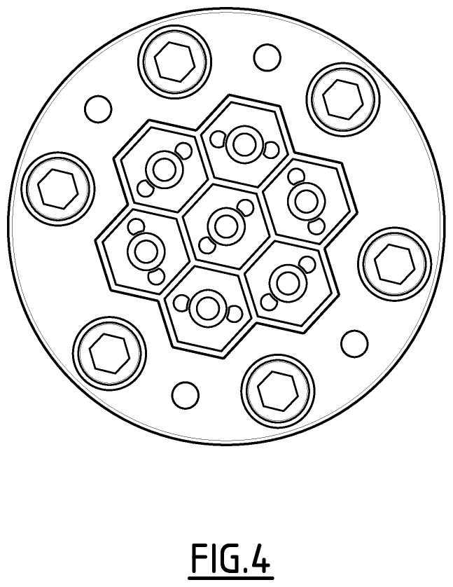

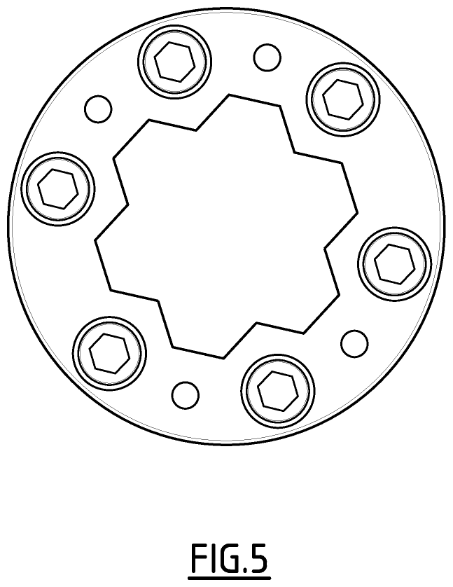

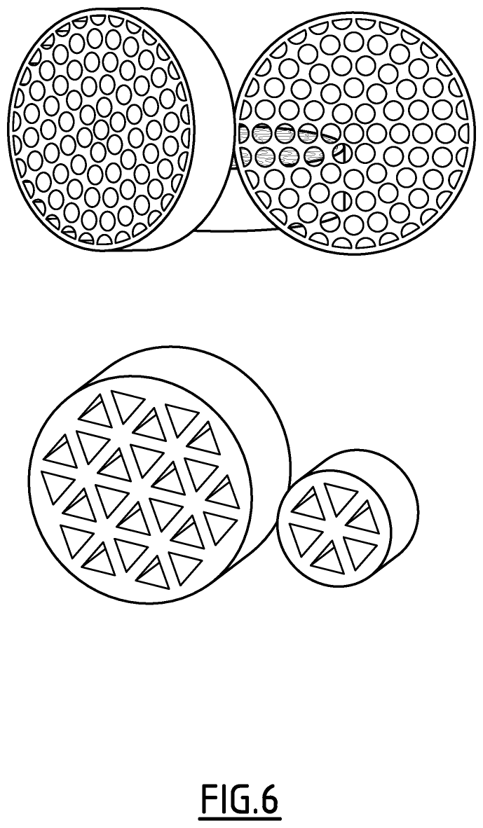

The mixture obtained at step 1 is passed through an extruder equipped in a first case with a die of the invention as illustrated in FIG. 4, and in a second case with a comparative die as illustrated in FIG. 5;
On leaving the die, the bars are respectively extruded and cut to obtain the weights and volumes given in Table 1 below.
Solid single-dose care compositions are respectively obtained, in the form of a solid composition of the invention having a honeycomb structure (dose 1, similar to those illustrated for example in FIG. 6), and a comparative solid composition generally of the same shape of dose 1 but without a cavity (doses 2 and 3).

TABLE 2

| | Dose 1 | Dose 2 | Dose 3 |
|---|---|---|---|
| | (invention) | (comparative) | (comparative) |
| Weight (in mg) | 750 | 750 | 3000 |
| Volume (in cm³) | 3 | 0.75 | 3 |
| Porosity (%) | >75 | 0 | 0 |

### Step 3—Application:

Step 3 was conducted with a panel of 18 women aged between 22 and 47 years (hereafter the «candidates»), 3 groups being formed with each group comprising 6 women (groups A, B and C).

For each group, the candidates applied the tested composition to their hair after previously mixing the composition in the hands to obtain a viscous liquid solution.

Each of the groups A, B and C used doses 1, 2 and 3, but each time in a different order.

At this step 3, the candidates assessed the performance of doses 1, 2 and 3 in terms of (i) ease of prehension, (ii) homogeneity of the applied deposit, (iii) comfort on application and (iv) state of the composition after use.

### Scoring Criteria:

TABLE 3

| SCORING CRITERIA | 0 | 1 | 2 |
|---|---|---|---|
| Prehension | Satisfactory | Moderate | Poor |
| Homogeneity of deposit | Satisfactory | Moderate | Poor |
| Comfort on application | Satisfactory | Moderate | Poor |
| State of the composition | No residues | Some | General structure |
| after use | | residues | retained |

The averages of the results obtained with the three groups A, B and C are given in the table below, on the understanding that the same trends were observed between the three groups.

TABLE 4

| | Dose 1 | Dose 2 | Dose 3 |
|---|---|---|---|
| | (Invention) | (Comparative) | (Comparative) |
| Prehension | 0 | 2 | 0 |
| Homogeneity of deposit | 0 | 1 | 1 |
| Comfort on application | 0 | 1 | 1 |
| State after use | 0 | 1 | 2 |

With the comparative doses 2 and 3, it was observed that if the size is too small prehension is poor and handling is difficult, and if too bulky a single dose cannot be fully consumed, generating much loss.

In addition, doses 2 and 3 have moderate properties in terms of homogeneity of deposit and comfort on application.

Dose 1 of the invention allows combining of the advantages of comparative doses 2 and 3 without the disadvantages thereof.

A composition of the invention, through the presence of cavities in the contents under consideration provides access to solid hair care compositions combining the ability to generate a homogeneous deposit with less material while generating less loss and exhibiting satisfactory homogeneity and comfort on application, without being detrimental to handling properties.

A solid composition of the invention is less raw material-intensive and is therefore advantageous both economically and ecologically.

1. A solid cosmetic hair and/or scalp care composition comprising at least one hair care agent, wherein said composition comprises at least one cavity such that the composition has a porosity of at least 20% by volume relative to the total volume of said solid composition.

2. The solid composition according to claim 1, wherein the porosity is higher than or equal to 30%, preferably higher than or equal to 40%, in particular higher than or equal to 50%, even higher than or equal to 60%, in particular higher than or equal to 70%, and most particularly higher than or equal to 80% by volume relative to the total volume of said solid composition.

3. The solid composition according to claim 1, wherein the cavity/cavities are through cavities.

4. The solid composition according to claim 1, said composition comprising one or more cavities, preferably several cavities, the same or different.

5. The solid composition according to claim 1, wherein the shape of the cavity/cavities is round, oblong, ovoid, triangular, polygonal, honeycomb or of tessellated shape (or partitioned) for example of Voroni diagram type, preferably of honeycomb shape.

6. The solid composition according to claim 1, wherein the ratio between the volume of the cavity/cavities and the total volume of the composition is between 0.2 and 0.9, preferably between 0.2 and 0.8, better still between 0.2 and 0.7, even between 0.2 and 0.6.

7. The solid composition according to claim 1, wherein the hair care agent is chosen from among surfactants; fixative polymers; conditioning agents; fats; polysaccharides; anti-UV agents; fillers; fragrances; peptizing agents; acidic agents; alkaline agents; reducing agents; oxidizing agents; direct or oxidative dyes; and mixtures thereof.

8. The solid composition according to claim 1, wherein said composition comprises between 0.0001% and 60%, preferably between 0.001% and 50%, in particular between 0.01% and 40%, better still between 0.1% and 35%, and preferably between 1% and 25% of hair care agents relative to the total weight of said composition.

9. The solid composition according to claim 1, wherein said composition comprises at least one lipophilic gelling agent, preferably heat-sensitive, at least one oil, and mixture thereof.

10. The solid composition according to claim 1, wherein said composition additionally comprises at least one aqueous phase dispersed in the solid composition, preferably in encapsulated form.

11. The solid composition according to claim 1, wherein said composition is a single-dose composition.

12. A kit comprising:
at least one first solid composition according to claim 1;
at least one second liquid composition; and
optionally, at least one container.

13. Packaging comprising several solid composition according to claim 1, preferably in single-dose form, said compositions comprising hair care agents and/or having contents of different hair care agent(s).

14. A process for manufacturing a solid cosmetic composition according to claim 1, comprising at least the steps of:
(i) providing a composition in liquid or semi-liquid form;
(ii) pouring the composition into a mould and drying until a solid composition is obtained;
(iii) cutting the solid composition to the desired shape; and
(iv) forming cavities to obtain the solid composition.

15. A process for manufacturing a solid cosmetic composition according to claim 1, comprising at least the steps of:
(a) providing a composition in liquid or semi-liquid form;
(b) passer the composition of step (a) through an extruding device equipped with a die having a shape adapted to form a solid bar; and
(c) cutting the bar obtained at step (b) to obtain the solid composition.

16. A non-therapeutic cosmetic hair and/or scalp treatment method comprising at least one step to apply to the hair and/or scalp at least one composition according to claim 1.
